(19) United States
(12) Patent Application Publication
(10) Pub. No.: US 2005/0106258 A1
(43) Pub. Date: May 19, 2005

(54) Pharmacology and manufacturing method of Bombyx moriL related bioproduct

(76) Inventors: Jiao Gong, Arcadia, CA (US); Wei Jiang, Arcadia, CA (US)

Correspondence Address: JIAO GONG, 411 S. 3RD AVE #B, ARCADIA, CA, 91006, US

(21) Appl. No.: 10/713,700

(22) Filed: Nov. 17, 2003

(51) Int. Cl. A61K 35/78; A61K 35/64

(52) U.S. Cl. 424/538; 424/739; 424/773; 424/728; 424/757; 424/776; 424/777

(57) ABSTRACT

The reach of R. E. Furchgotte and L. J. Ignarro which leads to the “NO theory” has shock the whole world. My invention is a combined patent including pharmacology as well as isolation and purification of Bombyx moriL. In my product, Bombyx moriL accounts for 56%, others 44%. We also adopt WLD resin absorption, other purification technology, and gas chromatography The biological activity is ensured because all the process is at 85° C. My product has selective effect on cavernous body, increasing cGMP and NO by the inhibition in of PED₅ enzymes. The Doppler test for the maximum and average blood flow in cavernous body also further proved the conclusion of the pharmacological activity.

### BACKGROUND OF THE INVENTION

Patented invention of pharmacology and manufacturing method of Bombyx moriL related bioprodcut: This product is composed of 56% Bombyx moriL and 44% Chinese herbs, which is processed by ethanol immersion, reflux extraction, concentration, elution, spray drying at 85° C. The quality of our product is guaranteed by the component testing of gas chromatography the results from pharmacological tests of animal model indicated that our product has specific effect on NO synthesis and metabolism in the cavernous body of the pennies, inhibiting PED₅ enzymes, reducing cGMP degradation, increasing cGMP concentration. The increased cGMP concentration leads to NO accumulation which contributes to the improved maximum and average blood flow in the cavernous body of penis. With Viagra as the standard control, the pharmacological experiment indicates that our product has dual effects (table 5) and excel in some parameters. Our invention (the pharmacology and manufacturing method of Bombyx moriL can be categorized into the specific item of American Patent Act—Pharmacology and Manufacturing Method of Isolated and Purified Natural Product. Because its pharmacological activity meets the two requirements-unexpected and unknown discovery or application, our product is consistent with the issue 101 and issue 103 of American Patent Act.

In the case of Nelson V. Bowler 626F.2d 853, 206 USPQ 881,883 (CCPA 1980), Court of Appeals for the Tariff and Patent said “it is good for the public to know something about the pharmacological activity of any compound”. It is much quicker and easier to cure disease or at least alleviate the symptoms if the pharmacological activity of a certain compound is known to the doctors. It is important to encourage the researchers in order to publicize more pharmacological activities of compound. The detailed explanations of the treatment or pharmacological practicality can be found in the Examination Guidelines of United Patent and Trademark office as well as the judgment of the court. The basis of the practicality evaluation is the pharmacological activity in the treatment and prevention.

www.house.gov/Judiciary/dick0713, in the first case involving the vitamin B₁₂, a natural compound (1958), the No4 court of Appeals for the Federal Circuit said “if it is a new and useful compound, the patentability of the natural product should not be denied by the Patent Act (1952). In term of the natural raw material, all the things protected by Patent Act are natural. The court went a step further” The Fact that the new and useful product is the result of isolation, concentration and purification of the natural raw material should not be used to deny its patentability.

Inre Bergy, 563F, 2d, 1031, (1977) 596F, 2d, 952, 201, U, S, P, Q, 352. According to the judgment of American Court of Appeals for the Tariff and Patent in 1979, the biologically purified product of bacterial culture is patentable. Because the purified form doesn't exist in nature, it comes into being under the well controlled situation in the lab. In the patent practice of Armenia, the courts assert that the isolated and purified natural product is patentable. The Bombyx moriL related bioproduct, an isolated and purified natural product as well as a new and useful compound, conforms to the judgment principles of the American courts.

R. F. Furchgotte and L. J. Ignarro, two American scientists discovered that NO was a magic molecule in the control of vasodilation. NO can have direct effect on GC which catalyzes AGP into cGMP. cGMP can relax the smooth muscle cell and dilate blood vessels. The two American scholars won the Nobel Prize in 1998 for their contribution in establishing the “NO Theory”.

American common law: 103-417. Food Additive Act (1994) defined the food additive as vitamin, mineral herb and other medical plant as well as their concentrate, metabolite, structural component and other synthesized product. According to regulations on the nutrient description, the effect of the nutrient or diet on the human structure and function should be defined, as well as the characteristics or benefits. The Bombyx moriL related bioproduct, as a whole, has wonderful pharmacological activity. After complex and strict purification and extraction process, it becomes a useful new product with pharmacological activity of treatment and prevention. For that reason, our product is consistent with American Patent Act.

### BRIEF SUMMARY OF THE INVENTION

R. F. Furchgotte and L. J. Ignarro won the Nobel Prize in 1998 for establishing “NO theory”. Taking advantage of this theory, a large American pharmacy, Pfitzer has produced Viagra a compound which can dilate the cavernous body of penis by inhibiting PED₅. As the first synthesized drug for erectile dysfunction (ED), Viagra is very popular around American or even in the world. However, Viagra has serious side effect on the cardiomuscular ischemia which causes some patients with heart disease cannot take this drug until it is quite necessary. It is a flaw of this wonderful drug.

Our product adopts 14 kinds of natural herbs and medical insects which is processed by strict isolation and purification. The source of the enzymes is similar with the condition in vivo, and it has been proved that it can affect the enzymes in the body.

The penis is composed of three cylindric masses, one among which is called cavernous body (its length is greater than the width). The cavernous body is full of the soft, sponge-like arteries which are responsible for the erection. Normally, the cavernous arteries are flat due to the control of numerous smooth muscle cells which prevent the excessive blood flow into the penis. If the smooth muscle cell is out of control, the erection may occur at any time and any place. Around the cavernous body, there is white fibrous membrane called albugineous coat which control the blood direction under the condition of erection—only flowing in, not out. Thus he erectile duration is ensured. Based on “NO Theory”, scientists discovered the physiological mechanism of erection, namely, the cavernous body in erection synthesizes and release NO, activating GC which catalyzes AGP into cGMP. cGMP can relax smooth muscle cell for vasodilation, increase penis blood flow and ensure a longer erection. (Table 4).

The Bombyx moriL related bioproduct contains inhibitor of PED₅. Just like Viagra, it can dilate cavernous arteries and increase the cavernous blood flow for longer erection by inhibiting the cGMP degradation and increasing cGMP level. In the experiment of male rats, our product increased the concentration of cGMP significantly. With the equal dose of Viagra and our product given to the animal tested respectively for 1.5 hours, both Viagra and our product group had higher cGMP concentration in contrast to the control, and our product group showed higher cGMP concentration in the cavernous body than that in the blood which indicated that our product had specificity and selectiveness for cavernous artery dilation. Furthermore, cGMP concentration of our product group was higher than that of Viagra group. The Bombyx moriL related bioproduct has wonderful result in the pharmacological activity test. It is really unexpected and our product is of great value in the treatment and prevention of disease.

### BRIEF DESCRIPTION OF THE SEVERAL VIEWS OF THE DRAWING

Table 1 The names of medicinal plants and insects from which the Bombyx moriL related bioproduct is made.

Table 2 Supplement facts of Bombyx moriL related bioproduct

Table 3 Manufacturing method and process flow.

Table 4 Physiological Mechanism of Erection

Table 5 Pharmacology of Bombyx moriL related bioproduct

Table 6 Mechanism of Viagra

Table 7 Cavernous Tissue of Rat

Table 8 Cavrenous Body of Rat

Table 9 Corpora Cavermosa of penis in male Rat

Table 10 Contrasting study on the pharmcay ecological Effects

Table 1 The names of medicinal plants and insects from which the Bombyx moriL related bioproduct is made. (see attached table 1 )

Table 2 Supplement facts of Bombyx moriL related bioproduct (see attached table 2)

Table 3 Manufacturing method and process flow. (see attached table 3)

Table 4 Physiological Mechanism of Erection(see attached table 4)

Table 5 Pharmacology of Bombyx moriL related bioproduct(see attached table 5)

Table 6 Mechanism of Viagra(see attached table 6)

Table 7 Cavernous Tissue of Rats

### Experiment and Research for Influence of Product Mainly Consisting of Bombyx moriL on 5′ Nucleotide Phosphodiesterase is Enzyme V in Corpus Cavernous Tissue of Rats

### SUMMARY

Phosphodiesterase together with cycloenzyme controls the level of cyclonucleotide in cell. It is reported from the document that after orally taking the Viagra, it has the function to selectively suppress Phosphodiesterase is enzyme, even block up the C-GMP, and degraded into 5′-GMP, but accumulating of C-GMP is the main link to lead to erection physiological function. According to the above pharmacology effect mechanism, we selected Viagra as positive drug, and wholly observed for effect of product mainly consisting of bombyx moriL on the Phosphodiesterase isoenzyme change in rats corpus cavernosum tissue, and the result is as shown below:

1. An hour later after drug was fed, took the corpus cavernosum tissue to check on Phosphodiesterase isoenzyme, and found that the samples of every group presented two strong bands with basically equal mobility with great gap between them, a few samples presented the third band, in which most of the comparison group samples had three bands, amony most of another test groups, only individual sample presented three bands. It is reported by the document that the viagra has the suppressing effect on hosphodiesterase isoenzyme V By inference, the test result might show that the test drug was inditical with the positive drug viagra and had the suppressing effect on the hosphodiesterase isoenzyme V. Due to this suppressing function, so only a few samples of every test group presented the third band, so that we could not conduct statistics. See FIG. 2-6.

2. According to the test result, we found that both product mainly consisting of bombyx moriL and viagra all had the suppressing effect on hosphodiesterase is enzyme. We had conducted statistics on the peak area of the second band, and the result indicates that, as the product mainly consisting of bombyx moriL high dose group, viagra middle and high dose groups in comparison with the comparison group, all have a difference, P<0.05. The product mainly consisting of bombyx moriL high dose group is better than viagra middle dose one. The product mainly consisting of bombyx moriL middle dose group also has the suppressing effect, but less. The suppressing effect of the test group on the first band is not clear. During test, only one sample presented the fourth band, but statistics were not done for it. The test result of us will provide the method how to more rationally use the product mainly consisting of bombyx moriL and explain its phyrmacological effect to the clinical doctors for reference.

### Material and Method

1. Main Agent and Equipment

1. 5′-[5-iodine indoxyl-(3)] thymidine acid ammonium salt

2. Polyacrylamide

3. Methylene Diacrylic Amine

4. Electrophoresis Power Supply

5. Laser Scanner

6. Circular Disk Electrophoresis Apparatus

II. Test Method

1. Animal Groups and Dose

Take 50 healthy male rats with body weight of 230±10.4 g, divide them into five groups at random, with 10 rats each group.

(1) The product mainly consisting of bombyx moriL test drug group: 200mg/kg and 400mg/kg two dose groups

(2) The positive comparison drug viagra group: 6 mg/kg and 12 mg/kg two dose groups

(3) The blank comparison group: normal saline 0.4 ml/per animal

Preparation of drug: After product mainly consisting of bombyx moriL and viagra are dissolved with quanitative normal saline, then use them.

Drug feeding way: Feed medicine to all groups of animal once.

2. Preparing of Corpus Cavernosum Sample

Take corpus cavernosum 10 mg, put into the tissue lapping homogenate container, add 0.05M PH7.4 phosphoric acid buffering agent liquid of 0.2 ml homogenate into the ice bath, and lastly add to 10 ml to prepare homogenate liquid with concentration of 1 mg/n ml. Centrifuge by 20 min. at temperature 4° C., 3000 r/min, then take the supernatant liquid for use.

3. Enzyme Content Measuring Principle

(1) Take 5′ Nucleotide Phosphodiesterase in the amount enough to dissolve phosphoric acid dieter bond by water, under the effecting of the bottom substance of 5′-[5-iodine indoxyl-(3)] thymidine acid ammonium salt, get iodine sub-indoxyl, it can be oxidized in air and generating violet 5.5′,by using -diuodoindigo principle to examine enzyme activity. After Electrophoresis treatment, keep the gel temperature in the bottom substance liquid to develop out the isoenzyme band.

4. Polyacrylamide Electrophoresis: It is Conducted Mainly According to the Reformed Thou Method

(1) Preparation of Polyacrylamide gel

Polymerize polyacrylamide monomer with cross-linking agent methylene diacrylic amine, under the acting of catalyst, they will polymerize and cross linked into gel. Grind evely and store the Polymerize polyacrylamide and methylene diacrylic amine according to 95:5 (w/w). When preparation, the total concentraction of the gel T=7%, the cross-linking agent percentage G=5%, dissolve in boric acid buffering liquid Tris of 0.09 m. PH9.0, exhaust air by use of a water pump several minutes, add tetramethylethyl-enediamine (TEMED) of 1% into 50 ml filtered solution and the new prepared fresh ammonium persulfate 10% will be mixed according to 0.8 ml.

Select a glass tube of 0.6×12 cm, add liquid into each tube quickly to reach height of 8 cm, then drop and add distilled water about 5 mm water layer around the tube wall at the gel surface by use of a narrow mouth dropper. Between the gel liquid and the water layer is formed a even and tidy interface, it indicates Polymerization is completed. The Polymerizing needs about 20 min at 20 C.°.

(2) Electrophoresis Method:

Add serum 30 μl via a micro injector, and add 0.01% bromphenol blue 20 μl containg saturated sucrose. After mixing evenly, slowly add the above said—Tries boric acid buffering liquid until flush with the glass tube opening. Put the gel tube into the bath-shape electrophoresis apparatus, each tube shall vertically stand. add the above mentioned Tries boric acid buffering liquid about 500 ml in the upper and lower electrode baths. Conduct electrophoresis at 4 C.°. The electrophoresis conduction: current strength 0.5 mA/tube, voltage 220V, time 1.5 hours. After electrophoresis is ended and the gel tube is degummed, immerse the gel into the bottom substance solution, i.e. dissolve 0.3 mg of each 5′-[5-iodine indoxyl-(3)] thymidine acid ammonium salt into 1 ml 0.05M PH 8.5 Tris-Hcl buffering liquid, and keep at 37 C.° constant temperature for a night, then conduct observation.

(3) Isoenzyme Observation Standard

At the temperature-cultured gel, there occured violet enzyme bands at different positions. measure the isoenzyme band number and the relevant position, and measure the distance from gel cylindrical surface to the mobile front edge via a laser scanner.

### Test Result

1. According to the gel column presenting violet enzyme bands at different positions, measure the band number of the isoenzyms by use of a laser scanner. It shows that most of the comparison group samples present three bands, among the other groups, only 1-2 samples present three bands. It is reported from the document, that viagra has the suppressing function on the Phosphodiesterase isoenzyme V. By reference, the third band of each group is caused by suppressing effect of drug on Phosphodiesterase isoenzyme V, but the comparison group could not be suppressed. This result indicates both product mainly consisting of bombyx moriL and viagra all have the suppressing effect on is enzyme V.

(1) In accordance with the measured result via the laser scanner, it shows that each group sample has two stronger is enzyme bands with a little difference in their position, and the mobility of each group sample is as shown in Table 1.

Table 1 is Enzyme Band Position of Each Group Sample(See Attached Table 7)

II. The second band of the phosphodiesterase is enzyme in the corpus cavernosum tissue for each test group rats has a certain difference in comparison between all groups. After the peak area of the gel column was scanned by laser, and calculate its area, the result indicates that as compared with viagra middle and high dose groups and product mainly consisting of bombyx moriL high dose group with the comparison one, they all have a difference P<0.05, and the result is as shown in Table 2.

Table 2 is Enzyme Band II Area Under Peak for All Groups Samples (See Attached Table 8)

III. Gel Column Scanning Diagram of Each Group Sample

1. From the gel column scanning diagram of the comparison group, it can be seen that among the peaks 1, II and III, the gap between peaks I II is larger with equal strong or weak degree, and the areas under the peaks is 0.56±0.21 and 0.495±0.318 respectively, and the area under the peak III is 0.379±0.06. See FIG. 1.

FIG. 1 Gel Column Scanning Diagram of Comparison Group Sample (See Attached 9)

2. From the gel column scanning diagram of viagra middle dose group, it can seen that two is enzyme peaks occurred in the figure, their position is the same as that of the comparison group, but the area under the peak II is obviously less than the comparison group P<0.05, See FIG. 2.

FIG. 2 Gel Column Scanning Diagram of Viagra Middle Dose Group (See Attached Table 10)

3. From the gel column scanning diagram of viagra high dose group, it can seen that the area under the peak II, amony the three speaks, has a difference in contrasting with the comparison group after statistics treatment, with P<0.05, See FIG. 3.

FIG. 3 Gel Column Scanning Diagram of Viagra High Dose Group(See Attached Table 11)

4. From the gel column scanning diagram of product mainly consisting of bombyx moriL middle dose group, it can seen that the peak II is shaper and higher, it has no difference in contrasting with the comparison group after statistics treatment, P>0.05, See FIG. 4.

FIG. 4 Gel Column Scanning Diagram of Product Mainly Consisting of Bombyx moriL Middle Dose Group (See Attached Table 12)

5. From the gel column scanning diagram of product mainly consisting of bombyx moriL high dose group, it can seen that the area under the peak II has a difference in contrasting with the comparison group after statistics treatment, P<0.05, its result is better than that of the viagra middle dose group. See FIG. 5.

FIG. 5 Gel Column Scanning Diagram of Product Mainly Consisting of Bombyx moriL High Dose Group(See Attached Table 13)

### SUMMARY

The above test results show that an hour later after the animal was fed with test drug, all the test groups have the suppressing effect on the rats phosphodiesterase is enzyme V in their coprus cavernosum, and the effect on another is enzyme displays different weak or strong. The result of the peak II indicates that as product mainly consisting of bombyx moriL and viagra high dose group is compared with the comparison group degree, all have a difference. The product mainly consisting of bombyx moriL high dose group is butter than viagra middle dose group.

### Main Reference Documents

K. C. Tsou, octal: Cancer Research 33: 2215-2217, 1973

Table 8 Cavernous Body of Rat's

### The Experimental Study on Product Mainly Consisting of Bombyx moriL's Effect on cGMP Content in Plasma and Cavernous Body of Rat's Pelvis

### SUMMARY

According to the aim of the experiment, IRA was performed to estimate cGMP content in plasma and cavernous boby of 50 male rats which were administrated by 2 dosages of viagra and product mainly consisting of bombyx moriL in our ladoratory. Change caused by viagra and product mainly consisting of bombyx moriL at 1.5 hours after administration was wholly observed.

Results:

1. Plasma Group: There was very significant difference (p<0.001) between viagra's middle and high dosage group and control group. There was significant difference (p<0.01) between product mainly consisting of bombyx moriL's middle dosage group and control group, while p<0.2 between product mainly consisting of bombyx moriL's high dosage control group.

2. Cavernous Boby Group: Contrasted with control group, difference of viagra's middle dosage group wasn't significant (p<0.1), while difference of high dosage group was remarkably significant (p<0.001). Difference of product mainly consisting of bombyx moriL's middle group was significant (p<0.01), while high dosage group was remarkably significant (p<0.001).

The experimental result provided a significant data to explain product mainly consisting of bombyx moriL's pharmacological action.

Materials and Methods

Materials:

I. Animals: 50 Wister rats,♂,235.50±12.50 g

II. Medicine and Reagent:

1. Product Mainly Consisting of Bombyx moriL'

2. Positive Control Medicine Viagra

3. CGMP RIA Kit

4. tea gents

Methods:

I. Rats: 50 healthy rats were randomly divided into 5 groups, every group is 10 rats

1. Product Mainly Consisting of Bombyx moriL Group: there were two dosage group 200 mg/kg, 400 mg/kg.

2. Viagra Group: two group, 6 mg/kg, 12 mg/kg.

3. Control Group: NS, 0.4 ML/rat.

Medicine Dispensing: Viagra and Product Mainly Consisting of Bombyx moriL was dissolved by certain mount of NS.

Administration Method: All Group was e.g. for One Time.

II. Abstract Method of Plasma Sample:

Anticoagulant 50 u l EDTA was added into blood test tube.lml blood was taken in rat's veniplex orbita posterior. Test tubes was stood in ice-water mixture and were centrifugated 200 rpm for 10 minutes. Then took 0.1 ml plasma, added 2 m1 absolute alcohol, mix 1 minute in rapid liquid mixer stood for 5 minutes, centrifugate 300 rpm for 10 minutes, took supernatant into little beaker, deposited by 1 ml 75% alcohol, repeated one time, combined supernatant of two times, dried it in oven under 60° C., prepared for usage.

III. Abstract Method of Cavernous Boby Sample:

Took 50mg cavernous boby, laid it in texture homogenization added 2 ml 50 mM PH7.4 sodium acetate buffer in ice-water mixture, took it into 10 ml test tube after fully mixing ,used 2 ml absolute alcohol to wash homogenate tube for one time, combined it with homogenate, stood 5 minutes, centrifugated 3500 rpm for 15 minutes, took supernatant into little beaker, deposited by 75% alcohol for two times, combined supernatant of two times, dried it in oven under 60° C., perepared for usage.

IV. Detecting cGMP in Plasma and Cavernous Boby:

Our laboratory used 125I-cGMP-RIA kit by Isotope Laboratory, detecting Method was double antiboby radioimmune method. The experiment sensitivity is 0.008 pmol/ml, most combination rate of detection is 82.7%,non wpecific combination rate is 5.0%.

### Experiment Result

1. Both cGMP content in rat's plasma of product mainly consisting of bombyx moriL's middle and high dosage group were higher than that of control group, which was the same as viagra As followed table 1:

Table 1 cGMP Content in Plasma Sample(See Attached Table 14)

1. cGMP content in rat's carvenous body of product mainly consisting of bombyx moriL's middle dosage group was significant different (p<0.01) from that of control group ,while there was remarkably significant difference (p<0.001) between high dosage group and control group, moreover cGMP content of high dosage group was higher than viagra's middle dosage group. AS followed Table 2.

Table 2 cGMP Content in Carvenous Boby(See Attached Table 15)

### Conclusion

1. Through above-mentioned results, we conclude that at 1.5 hours after administration of product mainly consisting of bombyx moriL, cGMP content in rat plasma of administrated group is remarkably higher than that of control group, and there is significant difference (p<0.01),while high dosage group is p>0.2.But cGMP content of both group were increased. Both middle and high dosage group of positive contrasting medicine viagra were remarkably significantly different from control group (p<0.00 1).

2. Effect on carvenous body of product mainly consisting of bombyx moriL's middle and high dosage group is much better than that on plasma. There are significant difference (p<0.01) and remarkably significant difference (p<0.001) between both product mainly consisting of bombyx moriL's middle and high dosage group and control group, meanwhile cGMP content of product mainly consisting of bombyx moriL's group is higher than that of viagra's middle dosage group.

The experiment showed that product mainly consisting of bombyx moriL and Viagra indeed had effect on making cGMP in rat's plasma and carvenous body increase, especially product mainly consisting of bombyx moriL have a stronger effect on cgMP in rat's carvenous boby than in plasma, which is identified with correlated references

### References

[1]S. Moncada, et al. Pharmacol Rev, 1991. 43(2): 110

Table 9 Corpora Cavermosa of penis in male Rat's

### The Influences of Product Mainly Consisting of Bombyx moriL on Nitric Oxide Contents in the Sera and the Corpora Cavernosa of Penis in Male Rats

### ABSTRACT

It has been approved in literatures that nitric oxide (NO) is formed in the endothelial cells in blood vessels and NO plays as an important signal transmitter in many physical activities of the human body. Nitric Oxide exists in many organs and tissues such as brain, blood vessels, liver, lung, pancreas, immune and reproductive systems. On the basis of No on wide-ranging regulative activities in the human physiological process, the Nitric Oxide contents, in the Sera and the Corpora Cavernosa of Penis in 50 normal male rats, were measured by nitrate reductase method for two dosages of product mainly consisting of bombyx moriL and Sildenafil Citrate(Viagra).On a half and one hour after the drug administrations, the variations of NO content were compared with the two drugs treated animals as a whole situation. The results are: (1) In the rat serum of the tested drug groups, there are distinct NO content differences with statistical signicance (p<0.01).When compared the No contents of the low dosage groups of the two drugs with the control group's, and there are very distinct NO content differences with statistical significance (p<0.001) in the high dosage groups of the two drugs. (2) In the corpora cavernosa tested drug groups, there is a distinct No content difference with statistical significance (p<0.01) in Sildenafil Citrate (Viagra) low dosage group and in other three groups (low, high dosage product mainly consisting of bombyx moriL and Sildenafil citrate (Viagra) high dosage groups).There are No content differences with statistical significance (p<0.05) when compared with the control group's. These results provide the theoretic basis on pharmacological actions of product mainly consisting of bombyx moriL.

### Experimental Materials and Method

1. Animals Preparation:

50 healthy male Wister rats weighing 235.5±12.5 grams

2. Tested Drugs and Reagents.

(1) Tested drug: Content was 200 mg per capsule

(2) Positive control drug: Sildenafil citrate (Viagra) tablets, The content of Sildenafil citrate was 100 mg per tablet.

(3) NO kits

(4) Reagents: All reagents used in the experiment were analytic reagents (AR).

### Method

1. Animal Groups and Drug Dosages:

50 healthy male rats were randomly divided by body weight into five groups, ten all tested groups when compared with the control group.

Nitric oxide contents in the corpora cavernosa of the tested rats(see attached table 16)

### Conclusion

1. According to the results of this study, in the different dosages product mainly consisting of bombyx moriL tested groups and a half and one hour after the administrations, the NO contents of sera of the rats are obviously higher than those in the control groups'. There are evident differences with statistical significance in low dosage group (p<0.01) and very obvious differences with statistical significance in high dosage group (p<0.001) when compared the NO contents of the negative control group. The results in product mainly consisting of bombyx moriL groups are equivalent to those in the Sildenafil citrate (viagra) groups.

2. In the corpora cavernosa groups, the NO contents of the samples in the low and high dosage groups of product mainly consisting of bombyx moriL are higher than those in the control groups', same as those in the Sildenafil citrate (viagra) groups.

3. The results show that product mainly consisting of bombyx moriL and Sildenafil citrate (Viagra) are indeed effective for increasing the NO contents in the sera and the corpora cavernosa of penis in male rats. These results are equivalent to the relevant documents reported by the others.

### Main Reference Documents

Nitric Oxide, In Physiology, Pathophysiology, and pharm and Exper Thera, Vol, 43(2)109.

Table 10 Contrasting study on the pharmacy ecological Effects

### Contrasting Study on the Pharmacological Effects of Product Mainly Consisting of Bombyx moriL and Viagra —the Influence on Arterial Blood Flow Rate of Corpus Cavernosum Penis of Normal Males

AIM: The physiological mechanism of penile erection expresses that NO is excreted in the cavernosum during sexual extraction, whose direct function is to activate GC, which catalyzes GPT to cGMP, so the smooth muscle of cavernosum is relax, and the inflowed blood volume is increased, then penis is erected. In this experiment, Doppler color-ultrasound instrument was used to observe the influence of product mainly consisting of bombyx moriL and viagra on the arterial blood flow rate of corpus cavernosum penis of normal males, to estimate the alternation of inflowed blood volume of cavernosum.

Tested Medicie: No.981005, light-blue capsule, 200 mg/capsule.

Sildenafilcitrate, viagra: light-blue rhombic table, 100 mg/tablet. The tablet was triturated to fine powder, and amylum was added. The tablet was triturated to fine powder, and amylum was added. After adequate mixture, the powder was filled into 200 mg capsule. (The same specification as product mainly consisting of bombyx moriL).There was 50 mg sildenafilcitrate in every 8 capsules.

Subjects: 46 normal males, age 23-29, provided.

Methods: Double-blind, auto-control and mutual control design. The influence of the two medicines on arterial blood flow rate of cavernosum was contrasted, Based on ages, the objects were randonmly divided into 4 groups, and there were 11-12 people in one group. Before administration, arterial blood flow of corpus cavernosum penis was estimated respectively by Doppler color ultrasound instrument (Acuson, American, 128×p/10 c),and blood pressure was also measured. Then 2 groups were respectively given high dosage Medicine, that is, 8 capsules product mainly consisting of bombyx moriL and 8 capsules of viagra, and the dose-effect relationship was observed. Blood pressure and cavernous arterial blood flow of every group were estimated respectively at I and 2 hours after administration, and the time-effect relationship of product mainly consisting of bombyx moriL and viagra was observed. Whether the alternation before and after administration and the difference between groups were significant was convinced here.

Tab. 1 The Influence of Product Mainly Consisting of Bombyx moriL and Viagra on Arterial Blood Flow of Corpus Cavernosum Penis of Normal Males(See Attached Table 17)

Results:

I. The Influence on Cavernous Arterial Blood Flow Rate of Normal Males.

1. The Influence on the Cavernous Arterial Maximal Blood Flow Rate

See as Tab.1, the cavernous arterial maximal blood flow rate of normal males(MAX)was 9.39±1.86 cm/s. For high dosage groups of product mainly consisting of bombyx moriL and viagra, contrasting the alternation before and at I hour after administration, MAX was increased remarkably, and the increasement was 73% and 59.2%. For low dosage groups of the medicines, the increasement was 50.5% and 46.6% respectively, and it was also remarkable. At 2 hours after administration, the enhancement to MAX of the two medicines was decreased. However, it was stil significant, contrasting with that before administration (P<0.05) (Char 1 and attached chart). There is no significant difference between the groups.(see attached table 18)

2. The Influence on the Cavernous Arterial Minimal Blood Flow Rate.

See as Chart 1, Cavernous arterial minimal blood flow rate of normal males(MIN)was 1.15±0.55 cm/s. For the high and low dosage groups of product mainly consisting of bombyx moriL and viagra, the enhancement was in difference degree at 1 and 2 hours after administration.

3. Influence on Arterial Average Blood Flow Rate of Cavernosum

The cavernous average blood flow rate of normal males(TAMX)was 2.57±0.96 cm/s. For the low and high dosage groups of product mainly consisting of bombyx moriL and viagra, at I hour after administration, TAMX was increased remarkably (P<0.01).The increasing rate was 44.9-46.8 and 51.5-53.6% respectively. At 2 hours after administratio, the increasement to TAMX was decreased, and by contrasting with TAMX before administration, the difference was not significant. (Tab.1, Chart2, and attached chart) Through comparison among groups, there's significant difference on the two medicine's increasement to TAMX. (see attached table 19)

4. The Influence on Pulsation Index of Cavernous Artery

Cavernous arterial pulsation index(PI)of normal males was 3.38±0.96, see as Tab. 1, except high dosage of product mainly consisting of bombyx moriL could remarkably increase PI at I hour after administration (P<0.01), all the other groups had no remarkable influence on PI(Chart 3 and attached chart).(see attached table 20)

II. The Influence on the Blood Pressure of Normal Males

See as Tab. 2. At 1-2 hours after administration, high dosage of product mainly consisting of bombyx moriL can remarkably decreased systolic pressure(sp)(p<0.01), and the decreasing rate was 4.6% and 4.9% respectively. For the high dosage viagra group, the decreasing rate was 6.2% (P<0.01)at 1 h after administration, and at 2 hours after administration, the difference wasn't significant by contrasting with that before administration. High dosage of product mainly consisting of bombyx moriL and viagra had remarkable hypotensive function only at I hour after administration, and the rate was 7.7% and 8.3% respectively. At 1-2 hours after administration, product mainly consisting of bombyx moriL and viagra could decreased diastolic pressure(DP)for different degree, but the difference was not significant by contrasting with that before administration.(Chart4).(see attached 21)

Tab. 2 The Influence of Product Mainly Consisting of Bombyx moriL and Viagra on the Blood Pressure of Normal Males(See Attached Table 22) of Normal Males

### Conclusion

No matter high or low dosage product mainly consisting of bombyx moriL and viagra, at 1 h after administration, the maximal and average blood flow rate of normal males cavernous artery could be increased remarkably. However, there is no remarkable influence on the minimal blood flow volume. At 2 hours after administration, the increasement of maximal blood flow rate was still remarkable. To PI, except that high dosage product mainly consisting of bombyx moriL had remarkable increasing effects at 1 h after administration, viagra had no remarkable influence on it. At I hour after administration, product mainly consisting of bombyx moriL and viagra had remarkable hypotensive effects, but the influence on DP was not significant, the hypotensive effects of high dosage product mainly consisting of bombyx moriL could lasting for 2 hours.

### DETAILED DESCRIPTION OF THE INVENTION

1. “Pharmacology and Manufacturing Method of Bomyx moriL related bioproduct” is the Patent of pharmacology as well as Isolation and purification of Natural Product. Our product adopts Bombyx moriL 56%; Rhizoma corydalis 6.5%; Fructus Schisandae 5.5%; Herba Epimeil 4.4%; Cortex Cinnamomi 2.2%; Tritonellia Gyaesin 3.5%; Semen Cuscutae 2.0%; Semen Alii Tuberosi 2.2%; Fructus Foeniculi 1.1%; Herba Cistanchis 1.1%; Common Panaxoside Ginseng 6.5%; Radix Achyranthis Bidentatae 4.4%; Rhizoma Curculiginis 3.5%; Fructus Cnidii 1.1% (table 1, 2). The process flow can be described as “for the Bombyx moriL: mixing every 100 kg Bombyx moriL with 200 kg 65% ethanol; soaking at 25° C. for 30 days; homogenizing and then soaking at 25° C. for 60 days; collecting supernatant and concentrating with reduced pressure at 85° C.; and keeping for spray-draying. For the medicinal plants: mixing 100 kg raw material with 300 kg 65% ethanol; soaking for 12 hours and reflux extraction at 85° C. for 3 hours; collecting filtrate; mixing dregs with 200 kg 65% ethanol, reflux extraction at 85° C. for 2 hours, then mixing the remaining dregs with 100 kg 65% ethanol, reflux extraction again at 85° C. for 1 hour; discarding the dregs. For the last step: collecting all the filtrate, concentrating at 85° C.; collecting supernatant; a absorbing through WLD resin affinity column; concentrating the elution at 85° C.; spray-drying at 85° C. together with the concentrate of the bombyx moriL; then the fine powder for the gas chromatography to ensure the quality.” (See Table 3)

2. Pharcological study: Our product contains PED₅ enzyme inhibitor. Ped₅ enzymes are bad for the microcirculation because they can degrade cGMP quickly. Our product resists cGMP degradation by inhibiting PED₅ enzymes which can lead to the vasodilation, increased blood flow in the reproductive organ and longer erection.” (Table 5, 7).

3. Pharmacological experiment: The cGMP concentration in the male rat's blood was higher than that of the control, 1.5 hours after administration. Furthermore, the concentration of penis was higher than that of blood which suggested that our product has selectiveness to the reproductive system for the vasodilation, improved microcirculation and enhanced nutrition.

4. Our product can increase NO concentration in the cavernous body. The normal value is 29.2 nmol/L, however, the NO concentration in the rat's cavernous body is as high as 42.3 nmol/L after administration, 1.4 times higher than normal. Our product can also increase NO concentration in blood. The normal value is 4.45 nmol/L, however the NO concentration in the rat's blood is as high as 29.01 nmol/L after administration, 6.5 times higher than normal. It suggests our product can improve microcirculation and nutrition supply of tissue and organs.

5. Color Doppler for Blood Flow Rate of Cavernous Body: the normal value of is maximum blood flow in the cavernous body is 9.39 cm/s, however, the result of 1 hour after administration measured by American Aclison 128×P/10C is 16.24 cm/s, increasing by 73%. The normal value of average blood flow in the cavernous body is 2.57 cm/s, however the result of 1 hour after administration is 3.89 cm/s, increasing by 51.5%. Our study suggests that the blood flow increases, and erection is longer which are consistent with Experiment 2,3,4(Table 10).

(attached table 1)

| 1 | Bombyx moriL | 2 | Rhizoma corydalis | 3 | Fructus Schisandae |
|---|---|---|---|---|---|
| | Saturniidae | | | | |
| Antheraea Pernyi | | Papaveraceae Corydalis | | Magnoliaceae Schisandra | |
| Gnerin-meneville (male adult) | | Yanhuso W. T. Wang (dry | | Chinensis(Tuncz)Baill (fruit) | |
| | | stem tuber) | | | |
| 4 | Herba Epimedii | 5 | Cortex Cinnamomi | 6 | Semen Trlgonellae |
| Berberidaceae Epimedium | | Lauraceae Cinnamomum | | Leguminosae Trigonella | |
| brevicoram Maxm (falling | | Cassia Presl (dry hide) | | foenum-graecuml (seed) | |
| branches) | | | | | |
| 7 | Semen Cuscutae | 8 | SemenAllii Tuberosi | 9 | Fructus Foeniculi |
| Convolvu laceae(cuscutoideae) | | LiLiaceae Allium | | Umbelliferae Foeniculum | |
| Cuscuta Chinensis Lam (fruit) | | tuberosum RottL. (seed) | | Vulgaremill (fruit) | |
| 10 | Herba Cistanchis | 11 | Common Panaxoside- | 12 | Radix Achyranthis- |

| | | |
|---|---|---|
| Orobanchaceae Cistanche | Ginseng Araliaceae Panax | Bidentatae Amaranthaceae |
| deserticola Y. C. Ma (succulent | Ginseng C. A. Mey (dry | Achyranthes bidentata BL. |
| stem) | root) | (dry root) |

| | | | |
|---|---|---|---|
| 13 | Rhizoma Carculiginis | 14 | Fructus Cnidii |

| | |
|---|---|
| Ainary llidaceae Curcudigo | Umbelliferae Cnidium |
| Orchioides Gaertn(root and | Monnieri(L.)Cuss.(fruit) |
| stem) | |

(attached table 2)
Total: 100%

| | |
|---|---|
| 1. Bombyx moriL | 56% |
| 2. Rhizoma Corydalis | 6.5% |
| 3. Fructus Schisandae | 5.5% |
| 4. Herba EpimeiL | 4.4% |
| 5. Cortex Cinnamomi | 2.2% |
| 6. Tritonelliae Gyaesin | 3.5% |
| 7. Semen Cuscutae | 2.0% |
| 8. Semen Alii Tuberosi | 2.2% |
| 9. Fructus Foeniculi | 1.1% |
| 10. Herba Cistanchis | 1.1% |
| 11. Common Panaxoside Ginseng | 6.5% |
| 12. Radix Achyranthis Bidentatae | 4.4% |
| 13. Rhizoma Curculiginis | 3.5% |
| 14. Fructus cnidii | 1.1% |

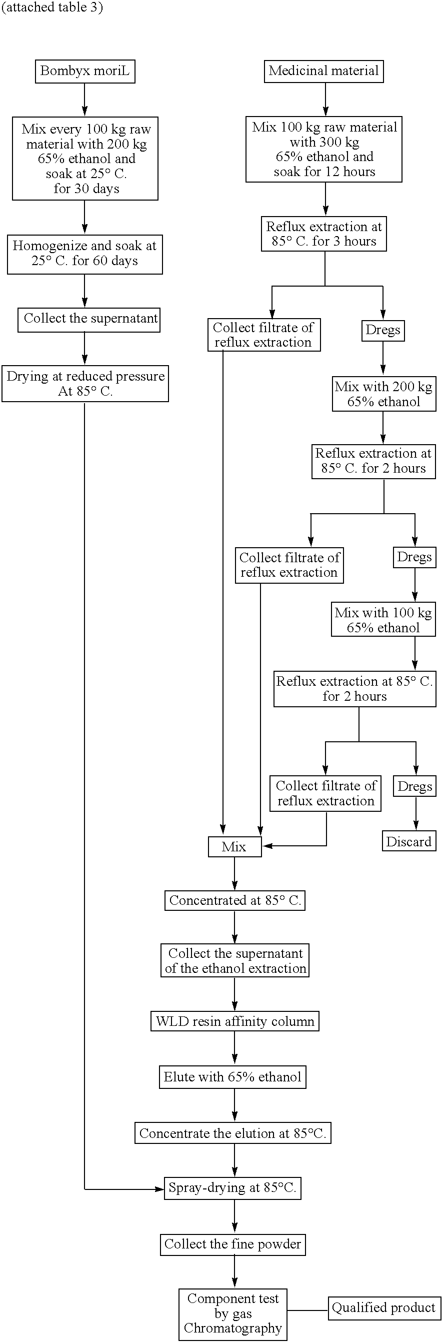 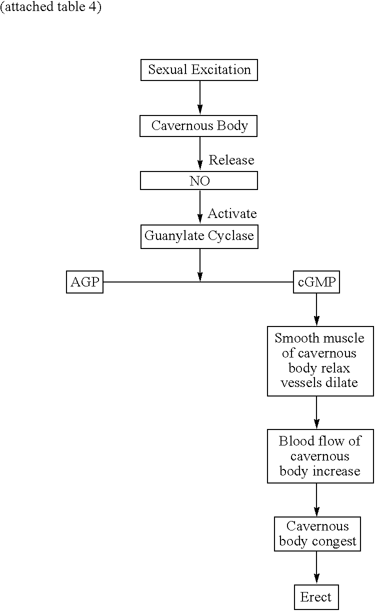 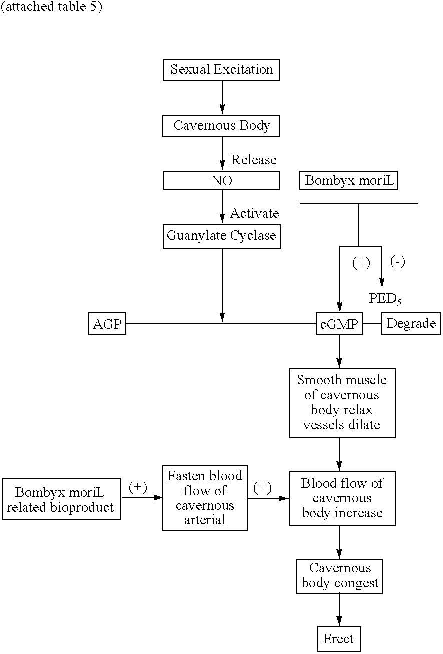 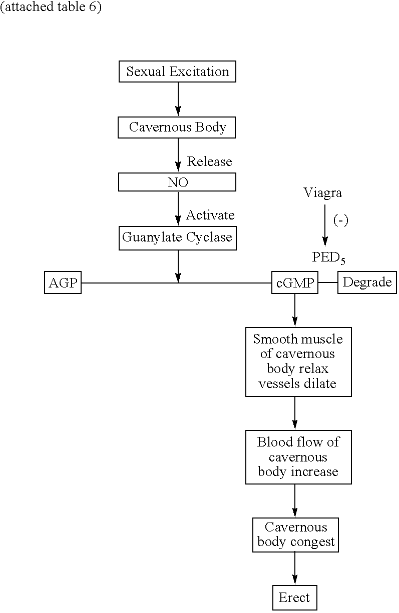 (attached table 7)

| | Number of Animal | Mobility of | Mobility of 2nd |
|---|---|---|---|
| Group | (n) | 1st. band X ± SD | band X ± SD |
| Blank Comparison | 10 | 0.21 ± 0.01 | 0.21 ± 0.01 |
| (Normal Saline) | | | |
| Viagra 6 mg/kg | 10 | 0.21 ± 0.01 | 0.1 ± 0.05 |
| Viagra 12 mg/kg | 10 | 0.21 ± 0.01 | 0.08 ± 0.02 |
| Product mainly consisting of | 10 | 0.23 ± 0.01 | 0.09 ± 0.01 |
| bombyx moriL 200 mg/kg | | | |
| Product mainly consisting of | 10 | 0.22 ± 0.01 | 0.08 ± 0.01 |
| bombyx moriL 400 mg/kg | | | |

(attached table 8)

| | Number of | Peak Area | |
|---|---|---|---|
| Group | Animal (n) | X ± SD | P Value |
| Blank Comparison | 10 | 0.495 ± 0.328 | |
| (Normal Saline) | | | |
| Viagra 6 mg/kg | 10 | 0.249 ± 0.126 | P < 0.05 |
| Viagra 12 mg/kg | 10 | 0.198 ± 0.092 | P < 0.05 |
| Product mainly consisting of | 10 | 0.306 ± 0.168 | |
| bombyx moriL 200 mg/kg | | | |
| Product mainly consisting of | 10 | 0.215 ± 0.521 | P < 0.05 |
| bombyx moriL 400 mg/kg | | | |

[image] [image] [image] [image] [image] (attached table 14)

| | Number of | | | |
|---|---|---|---|---|
| Group | Animals | Pmol/ml X ± SD | P | Remarks |
| Control Group(NS) | 10 | 2.59 ± 0.48 | | |
| Viagra Group 6 mg/kg | 10 | 4.53 ± 0.67 | P < 0.001* | Contrast with control group |
| Viagra Group 12 mg/kg | 10 | 4.42 ± 0.97 | P < 0.001 | Contrast with control group |
| Product mainly consisting of bombyx | 10 | 3.88 ± 1.01 | P < 0.01* | Contrast with control group |
| moriL Group 200 mg/kg | | | | |
| Product mainly consisting of bombyx | 10 | 2.80 ± 0.18 | p > 0.2 | Contrast with control group |
| moriL Group 400 mg/kg | | | | |

*Very significant difference
**Significant difference

(attached table 15)

| Group | Number of Animais | Pmol/ml X ± SD | P | Remarks |
|---|---|---|---|---|
| Control Group (NS) | 10 | 0.25 ± 0.05 | | |
| Viagra Group 6 mg/kg | 10 | 0.40 ± 0.26 | P < 0.1 | Contrast with control |
| | | | | group |
| Viagra Group 12 mg/kg | 10 | 0.53 ± 0.12 | P < 0.001 | Contrast with control |
| | | | | group |
| Product mainly consisting of | 10 | 0.45 ± 0.17 | P < 0.01 | Contrast with control |
| bombyx moriL | | | | group |
| Group 200 mg/kg | | | | |
| Product mainly consisting of | 10 | 0.43 ± 0.13 | P < 0.001 | Contrast with control |
| bombyx moriL | | | | group |
| Group 400 mg/kg | | | | |

(attached table 16)

| | | Anima | | |
|---|---|---|---|---|
| | Dosage | value* | NO contents | |
| Groups | (mg/kg) | Numbers | umol/L \overline{X} ± SD | P |
| Negative | 0.5 ml of | 10 | 29.2 ± 5.37 | |
| control | the | | | |
| | physical | | | |
| | saline | | | |
| | solution | | | |
| Viagra low | 6 | 10 | 44.88 ± 9.47 | P < 0.01 |
| Viagra high | 12 | 10 | 48.52 ± 17.95 | P < 0.05 |
| Product mainly | 200 | 10 | 41.98 ± 16.11 | P < 0.05 |
| consisting of | | | | |
| bombyx | | | | |
| moriL low | | | | |
| Product mainly | 400 | 10 | 42.36 ± 13.04 | P < 0.05 |
| consisting | | | | |
| of bombyx | | | | |
| moriL high | | | | |

*NOTE: When compared the NO contents of tested drug groups with the control groups.

(attached table 17)

| | | | Blood flow rate (cm/s, X ± SD) | | | | | |
|---|---|---|---|---|---|---|---|---|
| | | | MAX | | | MIN | | |
| | Case | Dosage | Before | 1 h | 2 h after | Before | 1 h after | 2 h after |
| Group | Number n | mg | administration | after administration | administration | administration | administration | administration |
| Product mainly | 11 | 800 | 9.18 ± 2.27 | +4.64 ± 2.25*** | +2.0 ± 2.19* | 1.09 ± 0.70 | +0.36 ± 0.67 | +0.36 ± 1.03 |
| consisting | | | | | | | | |
| of bombyx moriL | | | | | | | | |
| Product mainly | 12 | 1600 | 9.25 ± 1.42 | +6.75 ± 4.0*** | +3.33 ± 2.71** | 1.17 ± 0.58 | +0.17 ± 0.39 | +0.25 ± 0.62 |
| consisting | | | | | | | | |
| of bombyx moriL | | | | | | | | |
| Viagra | 11 | 25 | 9.55 ± 2.58 | +4.36 ± 2.98*** | +2.45 ± 3.78* | 1.09 ± 0.30 | +0.27 ± 1.01 | +0.36 ± 0.81 |
| Viagra | 12 | 50 | 9.58 ± 1.08 | +5.67 ± 4.31*** | +1.75 ± 2.63* | 1.25 ± 0.62 | +0.17 ± 0.72 | +0.17 ± 0.91 |
| | | | Blood flow rate (cm/s, X ± SD) | | | | | |
| | | | TAMX | | | PI, X ± SD | | |
| | Case | Dosage | Before | 1 h | 2 h after | Before | 1 h after | 2 h after |
| Group | Number n | mg | administration | after administration | administration | administration | administration | administration |
| Product mainly | 11 | 800 | 2.45 ± 1.29 | +1.09 ± 0.94** | +0.18 ± 0.98 | 3.47 ± 1.27 | +0.24 ± 1.24 | +0.32 ± 1.71 |
| consisting | | | | | | | | |
| of bombyx moriL | | | | | | | | |
| Product mainly | 12 | 1600 | 2.50 ± 0.67 | +1.17 ± 0.83*** | +0.42 ± 1.31 | 3.43 ± 0.74 | +0.87 ± 0.63*** | +0.45 ± 1.43 |
| consisting | | | | | | | | |
| of bombyx moriL | | | | | | | | |
| Viagra | 11 | 25 | 2.64 ± 1.12 | +1.36 ± 1.43* | 0.18 ± 1.17 | 3.24 ± 1.02 | −0.19 ± 1.16 | +0.10 ± 0.85 |
| Viagra | 12 | 50 | 2.67 ± 0.78 | +1.43 ± 1.27** | 0.50 ± 0.80 | 3.37 ± 0.79 | +0.24 ± 0.84 | −0.33 ± 0.72 |

Contrasted with amount before administration,
*P < 0.05,
**P < 0.01,
***P < 0.001;
“+, −” as increasement or decreasement

[image]

Chart 1 the influence of product mainly consisting of bombyx moriL and viagra on the cavernous arteral maximal blood flow rate of normal females

[image]

Chart 2 the influence of product mainly consisting of bombyx moriL and viagra on the cavernous arterial average blood flow rate of normal males

[image]

[image]

Chart 4 the influence of product mainly consisting of bombyx moriL and viagra on the blood pressure

(attached table 22)

| | | | Blood Pressure (mmHg, X ± SD) | | | | | |
|---|---|---|---|---|---|---|---|---|
| | | | Systolic Pressure | | | Diastolic Pressure | | |
| | Dosage | Case | Before | 1 h after | 2 h after | Before | 1 h after | 2 h after |
| Group | mg | number n | administration | administration | administrtion | administration | administration | administrtion |
| Product | 800 | 11 | 118.6 ± 5.9 | −9.1 ± 6.6** | +3.2 ± 6.0 | 81.4 ± 6.0 | −1.8 ± 5.6 | −1.4 ± 4.5 |
| mainly | | | | | | | | |
| consisting | | | | | | | | |
| of | | | | | | | | |
| bombyx | | | | | | | | |
| moriL | | | | | | | | |
| Product | 1600 | 12 | 120.0 ± 9.4 | −5.5 ± 6.0* | +9.5 ± 9.3** | 78.5 ± 10.5 | −2.0 ± 6.8 | −2.5 ± 6.3 |
| mainly | | | | | | | | |
| consisting | | | | | | | | |
| of | | | | | | | | |
| bombyx | | | | | | | | |
| moriL | | | | | | | | |
| Viagra | 25 | 11 | 120.5 ± 10.6 | −10.0 ± 6.3** | −3.5 ± 6.2 | 81.8 ± 6.4 | −2.7 ± 4.7 | −1.8 ± 4.6 |
| Viagra | 50 | 12 | 117.7 ± 9.8 | −7.3 ± 6.1** | −2.7 ± 5.6 | 78.6 ± 10.7 | −3.4 ± 7.2 | −2.0 ± 6.4 |

Contrasting with that before administraton,
*P < 0.05,
**P < 0.01,
***P < 0.001;
“+, −” expressed as increasement or decreasement.

1. My invention adopts Bombyx moriL as main component as well as other natural medicinal materials including: Bombyx moriL 56%; Rhizoma corydalis 6.5%; Fructus Schisandae 5.5%; Herba Epimeil 4.4%; Cortex Cinnamomi 2.2%; Tritonellia Gyaesin 3.5%; Semen Cuscutae 2.0%; Semen Allii Tuberosi 2.2%; Fructus Foeniculi 1.1%; Herba Cistanchis 1.1%; Common Panaxoside Ginseng 6.5%;Radix Achyranthis Bidentatae 4.4%; Rhizoma Curculiginis 3.5%; Fructus Cnidii 1.1%. The process flow can be described as “mixing 100 kg Bombyx moriL with 200 kg 65% ethanol; soaking at 25° C. for 30 days; homogenizing and soaking at 25° C. for another 60 days; collecting supernatant and concentrating with reduced pressure at 85° C.; keeping for the spray-drying; mixing 100 kg medicinal plant material with 300 kg, 200 kg and 100 kg respectively; reflux extraction at 85° C. and collecting all the filtrates; concentrating and collecting the ethanol supernatant; absorbing via WLD resin affinity column; eluting by 65% ethanol; spray-drying the elution as will as the supernatant of Bombyx moriL; and gas chromatography to ensure the quality.

2. I believe that the pharmacological experiments suggest my product can inhibit PED₅ enzyme, increase cGMP concentration in both the cavernous body and blood as well as the NO concentration. Furthermore, the color Doppler test for maximum and average blood flow also proves the conclusion. It is sure that my product is good for the treatment and prevention of disease.
